(19) United States
(12) Patent Application Publication
(10) Pub. No.: US 2012/0115175 A1
(43) Pub. Date: May 10, 2012

(54) METHODS FOR DIAGNOSING ELEVATED RIGHT OR LEFT VENTRICULAR FILLING PRESSURE

(75) Inventors: Navin K. Kapur, Hanover, MA (US); Richard Karas, Franklin, MA (US)

(73) Assignee: Tufts Medical Center, Inc., Boston, MA (US)

(21) Appl. No.: 13/288,493

(22) Filed: Nov. 3, 2011

Related U.S. Application Data
(60) Provisional application No. 61/410,464, filed on Nov. 5, 2010.

(51) Int. Cl. G01N 33/566

(52) U.S. Cl. 435/7.94; 435/7.92; 435/7.1

(57) ABSTRACT

The present invention features a method of diagnosing elevated left or right ventricular filling pressure and cardiovascular dysfunction in a subject by detecting increased levels of sEng in a biological sample from the subject.

### CROSS REFERENCE TO RELATED APPLICATIONS

This application claims benefit of U.S. Provisional Patent Application No. 61/410,464, filed Nov. 5, 2010, which is hereby incorporated by reference.

### BACKGROUND OF THE INVENTION

In general, the invention relates to the use of soluble endoglin (sEng) for the diagnosis of elevated left or right ventricular filling pressure and cardiovascular dysfunction.

Progressive right ventricular (RV) and left ventricular (LV) dysfunction induces expression of the cytokine transforming growth factor-β (TGFβ1). Endoglin (CD105) is a Type III TGFβ1 co-receptor that promotes binding of TGFβ1 and TGFβ3 to a Type II TGFβ-receptor. In vascular tissue, endoglin modulates downstream TGFβ1 signaling and regulates vascular tone. In cardiac tissue, endoglin is expressed by endothelial cells, fibroblasts in the connective tissue surrounding muscular fibers, and in fibroblast-like stromal cells of valve leaflets, while cardiac myocytes fail to demonstrate significant endoglin expression.

Right and left ventricular dysfunction are a major cause of global morbidity and mortality. Irrespective of the injurious mechanism, a decrease in right or left ventricular function limits the amount of blood ejected from the heart with each beat. As a result, residual blood volume accumulates in the right or left ventricle causing a corresponding increase in RV or LV pressure. Elevated right or left ventricular pressure is a characteristic of congestive heart failure.

There exists a need in the art for improved methods of diagnosing elevated left or right ventricular filling pressure and cardiovascular dysfunction.

### SUMMARY OF THE INVENTION

The present invention is directed to a method for diagnosing elevated left or right ventricular filling pressure by measuring sEng levels in a subject.

In one aspect, the invention features a method of diagnosing elevated left ventricular filling pressure (LVFP) in a subject by obtaining a biological sample from a subject and measuring the level of soluble endoglin (sEng) present in a biological sample, wherein an increase in the level of sEng in a subject compared to the level of sEng in a subject not suffering from elevated LVFP indicates elevated LVFP in a subject. The diagnostic method may also be performed by measuring the level of sEng in a biological sample taken from the subject, wherein an increase in the level of sEng in a subject compared to the level of sEng in a subject not suffering from elevated LVFP indicates elevated LVFP in a subject.

In a second aspect, the invention features a method of diagnosing elevated right ventricular filling pressure (RVFP) in a subject by obtaining a biological sample from a subject and measuring the level of sEng present in a biological sample, wherein an increase in the level of sEng in a subject compared to the level of sEng in a subject not suffering from elevated RVFP indicates elevated RVFP in a subject. The diagnostic method may also be performed by measuring the level of sEng in a biological sample taken from the subject, wherein an increase in the level of sEng in a subject compared to the level of sEng in a subject not suffering from elevated RVFP indicates elevated RVFP in a subject.

In certain embodiments, the methods of the invention may include a step of comparing the level of sEng in said sample to the a level of sEng is a sample taken from a subject not suffering from elevated LVFP or elevated RVFP.

In certain embodiments of any of the aspects of the invention, the level of sEng being measured is the level of sEng polypeptide or a fragment thereof.

In other embodiments, elevated RVFP or LVFP occurs in a subject with a cardiovascular condition or in a subject at risk of developing a cardiovascular condition. The cardiovascular condition may be acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, systolic or non-systolic heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, ischemic cardiomyopathy, and peripheral vascular disease.

The step of measuring in any of the aspects of the invention may include an immunoassay. The immunoassay may be an enzyme-linked immunosorbent assay (ELISA), radioimmunoassay, or immunofluorescence assay.

The methods of the present invention may further include measuring the level of one or more additional biomarkers in a biological sample. The one or more additional biomarkers may be brain natriuretic peptide (BNP), atrial natriuretic peptide (ANP), annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase.

In any of the methods described herein, the biological sample is blood, serum, or plasma.

In certain embodiments of the first of second aspect, the level of sEng in a subject not suffering from elevated left ventricular filling pressure or right ventricular filling pressure is between 1000-3500 pg/ml. And in certain embodiments, the level of sEng in a subject diagnosed with elevated left ventricular filling pressure or right ventricular filling pressure is between 3500-10000 pg/ml.

By “biological sample” is meant a bodily fluid (e.g., urine, blood, serum, plasma, or cerebrospinal fluid), tissue (e.g., cardiac tissue), or cell (e.g., cardiomyocyte) in which a polypeptide or nucleic acid molecule of the invention (e.g., sEng) is normally detectable.

By “biomarker related to a cardiovascular condition” is meant a biomarker that is known in the art to be derived from cardiac tissue and that is elevated in the circulation of subjects suffering from a cardiovascular condition. Exemplary biomarkers of a cardiovascular condition include, without limitation, brain natriuretic peptide (BNP), atrial natriuretic peptide (ANP), annexin V, β-enolase, cardiac troponin I, cardiac troponin T, creatine kinase-MB, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase, or markers related thereto. See, e.g., Scirica, J. Am. Coll. Cardiol. 55:1403-1415, 2010.

By “cardiovascular condition” or “cardiovascular dysfunction” is meant disorders of the heart and vasculature, including, for example, atherosclerosis, transient ischemic attack, systolic dysfunction, diastolic dysfunction, aneurysm, aortic dissection, myocardial ischemia, acute myocardial infarction (AMI), acute ST-segment elevation myocardial infarction (STEMI), acute non-ST-segment elevation myocardial infarction (NSTEMI), angina pectoris, unstable angina (UA), and stable angina (SA), myocardial infarction, congestive heart failure, dilated congestive cardiomyopathy, systolic or non-systolic heart failure, hypertrophic cardiomyopathy, restrictive cardiomyopathy, cor pulmonale, arrhythmia, valvular heart disease, endocarditis, pulmonary embolism, venous thrombosis, peripheral vascular disease, and peripheral artery disease.

By “elevated” is meant increased by at least 5%, 10%, 20%, 30%, 40%, 50%, 60%, 70%, 75%, 80%, 85%, 90%, 95%, or more.

By “expression” is meant the detection of a gene or polypeptide by methods known in the art. For example, DNA expression is often detected by Southern blotting or polymerase chain reaction (PCR), and RNA expression is often detected by Northern blotting, RT-PCR, gene array technology, or RNAse protection assays. Methods to measure protein expression levels generally include, but are not limited to, Western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), and flow cytometry, as well as assays based on a property of the protein including, but not limited to, enzymatic activity or interaction with other protein partners. Exemplary assays are described in detail in U.S. Patent Application Publication No. 2006/0067937 and PCT Publication No. WO 06/034507.

By “fragment” is meant a portion of a polypeptide or nucleic acid molecule that contains at least 5%, 10%, 20%, 30%, 40%, 50%, 60%, 70%, 80%, 90%, 95%, or more of the entire length of a nucleic acid molecule or polypeptide (e.g., sEng). A fragment may contain 10, 20, 30, 40, 50, 60, 70, 80, 90, 100, 200, 300, 400, 500, 600, or more nucleotides for sEng (SEQ ID NO: 1 and FIG. 10; NCBI Reference Sequence: NM_{—}000118.2). A fragment may contain 10, 20, 30, 40, 50, 60, 70, 80, 90, 100, 110, 120, 130, 140, 150, 160, 170, 180, 190, 200, or more amino acid residues for sEng (SEQ ID NO: 2 and FIG. 11).

By “left ventricular filling pressure (LVFP)” or “left ventricular end diastolic pressure (LVEDP)” is meant the pressure that builds in the left ventricle as the left ventricle fills with blood. Elevated LVFP is a consequence of abnormal left ventricular function and can result, for example, in the development of cardiovascular dysfunction or a cardiovascular condition.

By “reference sample” is meant any sample, standard, standard curve, or level that is used for comparison purposes. A “normal reference sample” can be, for example, a prior sample taken from the same subject or a normal healthy subject; a sample taken from a subject that does not have elevated left or right ventricular filling pressure or a disorder characterized by elevated left or right ventricular filling pressure; a sample taken from a subject that is diagnosed with a propensity to develop elevated left or right ventricular filling pressure, but that does not yet show symptoms of the condition; a sample taken from a subject that has been treated for elevated left or right ventricular filling pressure; or a sample of a purified reference polypeptide or nucleic acid molecule of the invention (e.g., sEng) at a known normal concentration. By “reference standard or level” is meant a value or number derived from a reference sample. A normal reference standard or level can be a value or number derived from a normal subject who does not have elevated left or right ventricular filling pressure. In certain embodiments, the reference sample, standard, or level may be, but need not be, matched to the sample subject by at least one of the following criteria: age, weight, body mass index (BMI), disease stage, and overall health.

By “right ventricular filling pressure (RVFP)” or “right ventricular end diastolic pressure (RVEDP)” is meant the pressure that builds in the right ventricle as the right ventricle fills with blood. Elevated RVFP is a consequence of abnormal right ventricular function and can result, for example, in the development of cardiovascular dysfunction or a cardiovascular condition.

By “soluble endoglin” or “sEng” is meant any circulating, non-membrane bound form of endoglin which includes at least a part of the extracellular portion of the endoglin protein and is substantially identical (e.g., at least 60%, 70%, 80%, 90%, 95%, 96%, 97%, 98%, 99%, or 100% identical) to the amino acid sequence encoding the extracellular portion of the endoglin protein. Soluble endoglin can result from the cleavage of the membrane-bound form of endoglin by a proteolytic enzyme. Soluble endoglin can also include circulating degradation products or fragments that result from enzymatic cleavage of endoglin and that maintain soluble endoglin biological activity. Preferred soluble endoglin polypeptides have soluble endoglin biological activity such as binding to substrates (e.g., TGF-β family members (e.g., TGF-β1 and TGF-β3) or TGF-β receptors (e.g., TβRI and TβRII)) or reversing or inhibiting angiogenesis by at least 10%, 20%, 30%, 40%, 50%, 60%, 70%, 80%, 90%, or more. Examples of assays for measuring these activities are known in the art and described in U.S. Patent Application Publication Nos. 2006/0067937 and 2005/0267021, and PCT Publication No. WO 06/034507, incorporated herein by reference. The term “soluble endoglin” also encompasses modifications to the polypeptide, fragments, derivatives, analogs, and variants of the endoglin polypeptide.

By “subject” is meant a human or non-human (e.g., bovine, equine, canine, ovine, or feline) animal. The methods described herein are applicable to both human and veterinary disease. Further, while a subject is preferably a living animal, the invention described herein may be used in post-mortem analysis. For example, the term “subject” encompasses living humans that are receiving or being evaluated for medical care, including persons with no defined illness who are being examined for signs of disease.

Other features and advantages of the invention will be apparent from the detailed description and from the claims.

### BRIEF DESCRIPTION OF THE DRAWINGS

FIGS. 1A-1C are a series of graphs showing soluble endoglin expression levels and LV filling pressure. When grouped by LVEDP, sEng levels are significantly higher in patients with an elevated LVEDP>16 compared to a low LVEDP<16 (4912±922 versus 3785±724 pg/mL, respectively, p<0.0001) (FIG. 1A). When grouped by LVEF, subjects with low LVEF<50% had significantly increased sEng levels compared to controls (4620±980 versus 3590±588, p<0.001, respectively) or subjects with LVEF>50% (4620±980 versus 3650±550, p<0.001, respectively) (FIG. 1B). When grouped by both LVEF and LVEDP, sEng levels were significantly increased in subjects with an elevated LVEDP, irrespective of LVEF (Group A versus B or D, p<0.001; Group A versus C, p=NS; Group C versus B or D, p<0.001; Group B versus D=NS).

FIG. 2 is a graph showing univariate regression plots for sEng (squares), ANP (triangles), and BNP (circles) (ANP: atrial natriuretic peptide; BNP: brain natriuretic peptide; sEng: soluble endoglin).

FIG. 3 is a graph with an accompanying table showing the predictive value of sEng as a determinant of elevated LVEDP. The receiving operator characteristic curve for sEng is shown with specific cut-points at various levels of sEng expression (NPV: negative predictive value; PPV: positive predictive value; Sens: Sensitivity; and Spec: specificity).

FIG. 4 is a graph showing that reduced sEng levels correlate with reduced pulmonary capillary wedge pressure (PCWP). Compared to baseline values, sEng levels were significantly reduced after 48 hours of diuretic therapy (*3830±330 versus 2540±1060 pg/mL, baseline versus follow-up, respectively, p<0.001) and corresponded with reduced PCWP (*19.5±3.1 versus 12.5+4.5 mmHg, baseline versus follow-up, respectively, p<0.001). Percent change in sEng was strongly associated with the percent change in LVEDP (R=0.75, p=0.008). In this group, levels of ANP and BNP were also reduced after diuretic therapy (ANP: 10.5±4.7 versus 5.2±1.9 ng/mL, respectively p=0.02; BNP: 8.8±2.5 versus 6.2±1.7 ng/mL, respectively, p=0.05).

FIGS. 5A-5C are graphs showing soluble endoglin expression levels in subjects with suspected LV dysfunction. Compared to healthy controls, sEng levels were significantly higher in the total group of 82 study subjects (3589±588 versus 4257±966 pg/mL, respectively, p<0.005; FIG. 5A). Among study subjects with a low LVEF<50%, sEng levels were increased regardless of whether the underlying etiology was non-ischemic or ischemic (FIG. 5B). Soluble endoglin levels were higher in subjects with ischemic cardiomyopathy compared to patients with non-ischemic heart failure (p=0.06). Among study subjects, worsening NYHA classification corresponded with increased sEng levels (NYHA Class 1: 3644.9±579, Class II: 4307±968, Class III: 4746±947, Class IV: 5089±1073 pg/mL; *p<0.01 versus Class 1; ANOVA p<0.001; FIG. 5C).

FIGS. 6A-6B are graphs showing the relationship between sEng expression and left ventricular filling pressure. FIG. 6A is a graph showing that sEng levels correlated directly with left ventricular end-diastolic filling pressure. FIG. 6B shows that, using a mouse model of cardiac pressure overload induced by thoracic aortic banding (TAC), endoglin mRNA expression was increased by 7-fold in the left ventricle and 14-fold in the left atrium compared to sham-operated controls 2 weeks after TAC.

FIGS. 7A-7B are graphs showing rapid increase in sEng release (after cyclic mechanical stretch) by enzyme-linked immunosorbent assay (FIG. 7A), followed by increased endoglin mRNA expression by quantitative real-time PCR (FIG. 7B).

FIG. 8 is a graph showing that actinomycin-D abolished stretch-induced endoglin mRNA expression with minimal effect on sEng release or endoglin protein expression. SB-431542 or anti-TGFβ1 antibody treatment partially attenuated endoglin mRNA expression, but failed to inhibit sEng release.

FIG. 9 is a Western blot and ELISA showing that ERK inhibition attenuated stretch-induced mEng protein expression and sEng release.

FIG. 10 is the mRNA sequence of endoglin (SEQ ID NO: 1; NCBI Reference Sequence: NM_{—}000118.2).

FIG. 11 is the endoglin peptide sequence (P17813[26-658] (SEQ ID NO: 2).

### DETAILED DESCRIPTION

We have discovered that soluble endoglin levels significantly correlate with elevated ventricular filling pressure and that sEng is a more sensitive and specific predictor of cardiac pressure overload than known biomarkers. Furthermore, our experiments suggest that sEng release in response to cardiovascular diseases involving mechanical stretch do not require transcription of the endoglin gene and may instead be mediated by post-translational mechanisms (e.g., proteolytic cleavage). Thus, sEng serves as a diagnostic indicator of elevated LVFP and/or RVFP and cardiovascular conditions associated with elevated LVFP and/or RVFP.

### Soluble Endoglin and Elevated Ventricular Filling Pressure

As described herein, the present invention features methods for diagnosing or assessing elevated left and/or right ventricular filling pressure and cardiovascular dysfunction by determining the level of soluble endoglin (e.g., sEng polypeptide or a fragment thereof) present in a subject.

Elevated ventricular filling pressure (i.e., left and/or right ventricular filling pressure) may occur in subjects that have a cardiovascular condition or a propensity of developing a cardiovascular condition. The cardiovascular condition may be, for example, acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, systolic or non-systolic heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, or peripheral vascular disease.

### Diagnostic Methods

A subject experiencing elevated ventricular filling pressure (i.e., left and/or right ventricular filling pressure) or a subject with a propensity to develop elevated ventricular filling pressure may show an alteration (e.g., an increase of 5%, 10%, 20%, 30%, 40%, 50%, 60%, 70%, 80%, 90%, or more) in the levels of a sEng polypeptide. In one example, an increase in sEng expression in a sample taken from a subject compared to a normal reference sample is indicative of elevated ventricular filling pressure or a risk of developing the same. sEng can include full-length sEng polypeptide, fragments or degradation products of sEng, the polypeptide bound to a substrate or ligand, or free (e.g., unbound) forms of the sEng polypeptide. Standard methods may be used to measure polypeptide levels in any bodily fluid including, but not limited to, urine, blood, serum, plasma, or saliva. Such methods include immunoassays, enzyme-linked immunoassays (ELISA), radioimmunoassays (RIAs), competitive binding assays, Western blotting using antibodies directed to sEng or fragments thereof (e.g., R&D Systems Antibody No. BAF1097 versus human endoglin ectodomain), and quantitative enzyme immunoassay techniques.

The level of a sEng polypeptide or antibody that binds sEng can be measured once, and the level may be compared to a control sample from a subject not suffering from elevated ventricular (i.e., left or right ventricular) filling pressure. In other embodiments, the level of a sEng polypeptide or antibody that binds sEng can be measured at least two different times from the same subject and an alteration in the levels (e.g., an increase by 10%, 20%, 30%, 40%, 50%, 60%, 70%, 80%, 90%, or more) over time is used as an indicator of elevated left or right ventricular filling pressure or a risk of developing the same.

In any of the diagnostic methods of the present invention, the level of, for example, sEng polypeptide or antibody that binds sEng may be measured to diagnosis or assess the risk of a subject developing elevated left or right ventricular filling pressure and, in turn, cardiovascular dysfunction. For example, the level of sEng polypeptide present in a subject diagnosed with elevated ventricular filling pressure may be, for example, 3500, 3600, 3700, 3800, 3900, 4000, 4100, 4200, 4300, 4400, 4500, 4600, 4700, 4800, 4900, 5000, 5200, 5400, 5600, 5800, 6000 pg/ml, or more, up to 10000 pg/ml, whereas the level of sEng in a subject not suffering from elevated ventricular filling pressure may be for example 3500, 3400, 3300, 3200, 3100, 3000, 2800, 2600, 2400, 2200, 2000 pg/ml, or less, as low as 1000 pg/ml. Diagnostic methods can include measurement of absolute levels or relative levels of a sEng polypeptide or antibody that binds sEng as compared to a reference sample. In one example, an increase in the level of sEng polypeptide or antibody that binds sEng as compared to a normal reference, is considered a positive indicator of elevated left and/or right ventricular filling pressure or a propensity to develop the same.

In other embodiments of the present invention, the biological activity of sEng may be measured, where an increase in activity relative to a sample taken from a control subject is diagnostic of elevated left or right ventricular filling pressure. For example, binding assays to measure sEng binding kinetics to sEng substrates (e.g., TGF-β family members (e.g., TGF-β1 and TGF-β3) or TGF-β receptors (e.g., TβRI and TβRII)) could be performed to quantitate sEng function. Other functional assays known to those skilled in the art could also be performed. See, e.g., U.S. Patent Application Publication Nos. 2006/0067937 and 2005/0267021 and PCT Publication No. WO 06/034507, incorporated herein by reference.

The diagnostic methods described herein can be used individually or in combination with any other diagnostic method for a more accurate diagnosis of the presence of, severity of, or predisposition to elevated left or right ventricular filling pressure in a subject. Such diagnostic methods include, for example, echocardiography, electrocardiography, coronary angiography, chest radiography, physical examination, histopathological examination, blood chemistry analysis, computed tomography, cytological examination, magnetic resonance imaging, and identification of other diagnostic biomarkers (e.g., brain natriuretic peptide (BNP), atrial natriuretic peptide (ANP), annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-Mb, S-100ao, myoglobin, actin, myosin, and lactate dehydrogenase). Left or right ventricular function may also be determined by measurement of various parameters such as the E/A ratio (early-to-atrial left or right ventricular filling ratio), the E (early left or right ventricular filling) deceleration time, and the isovolumic relaxation time.

### Diagnostic Kits

The invention also provides for a diagnostic test kit. For example, a diagnostic test kit can include polypeptides (e.g., antibodies that specifically bind to sEng or fragments thereof) and components for detecting and/or evaluating binding between the polypeptide (e.g., antibody) and sEng. Alternatively, the kit can include a sEng polypeptide or sEng fragment for the detection of sEng antibodies present in the serum or blood of a subject sample. In another example, diagnostic kits of the invention may be used to identify an alteration in the level of a sEng polypeptide relative to a reference, such as the level present in a normal control (e.g., the level in a subject not experiencing elevated left or right ventricular filling pressure). Such a kit may include a reference sample or standard curve indicative of a positive reference or a normal control reference.

For detection, either the antibody or the sEng polypeptide is labeled, and either the antibody or the sEng polypeptide is substrate-bound, such that the polypeptide-antibody interaction can be established by determining the amount of label attached to the substrate following binding between the antibody and the sEng polypeptide. Conventional immunoassays (e.g., ELISA) may be used for detecting antibody-substrate interactions and can be provided with the kit of the invention. The polypeptides of the invention can be detected in a biological sample, such as blood, plasma, or serum.

The diagnostic kit may include instructions for the use of the kit. In one example, the kit contains instructions for the use of the kit for the diagnosis of elevated left or right ventricular filling pressure or a risk of developing the same. In yet another example, the kit contains instructions for the use of the kit to monitor therapeutic treatment, dosage regimens, or subjects at risk of developing a cardiovascular condition.

### Subject Monitoring

The diagnostic methods described herein can also be used to monitor the onset of elevated left or right ventricular dysfunction in a subject during therapy or to determine the dosage(s) of therapeutic compound(s) needed to treat the condition. In this embodiment, the levels of sEng polypeptide or antibody that binds sEng may be measured repeatedly as a method of diagnosing elevated left or right ventricular filling pressure and also monitoring the treatment, prevention, or management of elevated left or right ventricular filling pressure and/or cardiovascular conditions associated with elevated left or right ventricular filling pressure. To monitor the progression of elevated left or right ventricular filling pressure in a subject, subject samples may be compared to reference samples taken early in the diagnosis of the disorder. In one example, levels of sEng polypeptide can be monitored in a subject that has been diagnosed with elevated left or right ventricular filling pressure. A decrease of sEng polypeptide in a subject being treated for elevated left or right ventricular filling pressure indicates an improvement in or the absence of elevated left or right ventricular filling pressure. Such monitoring may be useful, for example, in determining proper dosages for therapeutic treatment or in assessing the efficacy of a particular therapeutic regimen.

In addition, the diagnostic methods of the invention may be used to monitor a subject that has risk factors for cardiovascular diseases or disorders (e.g., a subject having a family history of a cardiovascular disease). In such an example, therapeutic methods can then be used proactively to promote vascular health and/or to prevent elevated left or right ventricular filling pressure from developing or further progressing.

### EXAMPLES

The present invention is illustrated by the following examples, which are in no way intended to be limiting of the invention.

### Example 1

### sEng Levels in Subjects with Elevated Left Ventricular Filling Pressure

We have discovered that soluble endoglin (sEng) levels are increased in association with elevated left ventricular (LV) filling pressure.

We enrolled 82 consecutive patients referred for evaluation of suspected LV dysfunction by right- and left-sided heart catheterization regardless of LV ejection fraction (LVEF) at Tufts Medical Center. Patients under 18 years of age and those presenting with an acute coronary syndrome, pregnancy, active or remote cancer, renal failure (estimated glomerular filtration rate ≦30), liver transaminases ≧2 times the upper limit of normal, non-sinus rhythm, or perceived interference with standard clinical care were excluded. All eligible patients who agreed to enroll had blood sampled at the time of arterial sheath insertion for diagnostic catheterization. LVEF was assessed by echocardiography or ventriculography at the time of catheterization. Data from the medical record and results of other blood tests were collected for subsequent analysis with SigmaStat 3.1 software.

To study whether sEng levels reflect diuresis-induced reductions in cardiac filling pressure during medical therapy for heart failure, we enrolled 10 patients with systolic heart failure as defined by a pulmonary capillary wedge pressure ≧16 mmHg and LVEF<50%, in whom follow-up pulmonary artery (PA) catheter measurements were deemed clinically necessary for hemodynamic monitoring after in-patient diuretic therapy. The same exclusion criteria listed above were applied to patients referred for follow-up right heart catheterization. Blood sampling was performed at the time of initial PA catheter placement and 48 hours after in-patient therapy. At each time point, sEng, brain natriuretic peptide (BNP) and atrial natriuretic peptide (ANP) levels were measured. Control subjects consisted of 25 healthy volunteers with no prior medical history, no active medical problems, and currently taking no medications as recorded by a screening questionnaire. Subjects were required to be between 21 and 80 years of age. Serum samples were obtained as a one-time lab draw in our clinical research center. Blood samples were collected using serum separator tubes and allowed to clot for 30 minutes prior to centrifugation at 2000×G for 15 minutes. Serum samples were immediately stored at −20° C. Human sEng, ANP, and BNP levels were measured in each serum sample in duplicate using commercially available quantitative sandwich enzyme immunoassay kits (sEng: R&D Systems; ANP and BNP: Phoenix Pharmaceuticals) according to the manufacturers' instructions. The intra-assay and inter-assay coefficient of variation for each protein assay were: sEng: 3% and 6%, respectively; BNP: 8% and 4%, respectively; and ANP: 11% and 5%, respectively.

All values are expressed as the mean and standard deviation (SD). Differences between means were detected by Wilcoxon rank-sum test with two-tailed p-values <0.05. Soluble endoglin data were normally distributed; however, because the natriuretic peptide data were not normally distributed, log BNP and log ANP were used in the correlations and regression models. Stepwise multiple regression analysis was performed to examine predictors of left ventricular end diastolic pressure (LVEDP) in left ventricular dysfunction (LVD). Variables entered into the model included age, sex, body surface area (BSA), hypertension, diabetes, hypercholesterolemia, active tobacco use, and history of myocardial infarction (MI). Stepwise multiple regression analysis was further performed to examine biomarkers as predictors of LVEDP in all patients. Variables entered into the model included: sEng, ANP, and BNP levels found in the serum of patients. Blockwise multiple regression, using the enter method, was also employed to examine sEng as a predictor of LVEDP after entering BNP, ANP, New York Heart Association (NYHA) functional classification, and LVEF into a single block. Stepwise multiple regression analysis was also performed to examine predictors of sEng. Variables entered into the model included: age, sex, BSA, hypertension, diabetes, hypercholesterolemia, active tobacco use, and history of MI. In a separate model, medication history was examined for the prediction of sEng levels. Medications entered into the model included: aspirin, beta-blockers, ACE inhibitors, calcium channel blockers, angiotensin receptor blockers, aldosterone antagonists, anti-lipidemics, and diuretics. To evaluate the value of sEng, BNP, and ANP measurements in the diagnosis of heart failure, we compared the sensitivity, specificity, and accuracy of each biomarker with measurements of LVEDP. Finally, we constructed receiver-operating-characteristic curves to illustrate various cutoff values of sEng, ANP, and BNP. A p-value <0.05 was considered significant. Statistical analyses were performed using SigmaStat software.

To determine whether sEng levels correlate with LVEDP, serum levels were measured by ELISA in 82 consecutive patients referred for cardiac catheterization to evaluate

LV filling pressures regardless of LVEF. The clinical characteristics of study subjects were categorized according to LVEDP and are presented in Table 1.

TABLE 1
Clinical characteristics of study subjects group by LVEDP

| | LVEDP (mm Hg) | |
|---|---|---|
| Variable | <16 (n = 47) | >16 (n = 35) |
| Age (years) | 55 ± 15 | 61 ± 12^{†} |
| Male | 26 (55%) | 27 (77%)^{†} |
| Body surface area (kg/m²) | 1.97 ± 0.26 | 2.05 ± 0.20 |
| Hypertension | 37 (79%) | 18 (53%)^{†} |
| Diabetes mellitus | 9 (19%) | 10 (29%) |
| Active smoking | 11 (23%) | 2 (6%)^{†} |
| Peripheral vascular disease | 2 (4%) | 4 (12%) |
| Coronary disease | 13 (28%) | 15 (44%) |
| Prior myocardial infarction | 8 (17%) | 11 (32%) |
| Cerebrovascular disease | 3 (6%) | 1 (3%) |
| NYHA class (Scale 0-4) | 1 | 3^{†} |
| LVEDP (mm Hg) | 10 ± 2.7 | 21.6 ± 4.4^{†} |
| LVEF (%) | 48 ± 16 | 25 ± 16^{†} |
| Medications | | |
| Aspirin | 33 (70%) | 17 (50%) |
| Clopidogrel | 9 (19%) | 3 (9%) |
| β-blocker | 28 (60%) | 18 (53%) |
| ACE-inhibitor | 18 (38%) | 13 (38%) |
| Calcium channel blocker | 5 (11%) | 3 (9%) |
| ARB | 1 (2%) | 1 (3%) |
| Aldosterone antagonist | 3 (6%) | 7 (21%) |
| Diuretic | 14 (30%) | 20 (59%)^{†} |
| Anti-dyslipidemic agent | 19 (68%) | 14 (40%) |
| Admission Lab Values | | |
| Sodium (mEq/L) | 139 ± 3 | 137 ± 3 |
| Creatinine (mg/dL) | 1.0 ± 0.8 | 1.5 ± 0.9^{†} |
| Blood urea nitrogen (mg/dL) | 17 ± 6 | 31 ± 18^{†} |
| Glucose (mg/dL) | 118 ± 30 | 131 ± 43 |
| WBC count (×10 cells/L) | 8.4 ± 3.3 | 8.1 ± 3.7 |
| Hemoglobin (g/dL) | 13.1 ± 1.5 | 12.6 ± 1.8 |

^{†}Significant group difference (p < 0.05); values are mean +/− standard deviation.

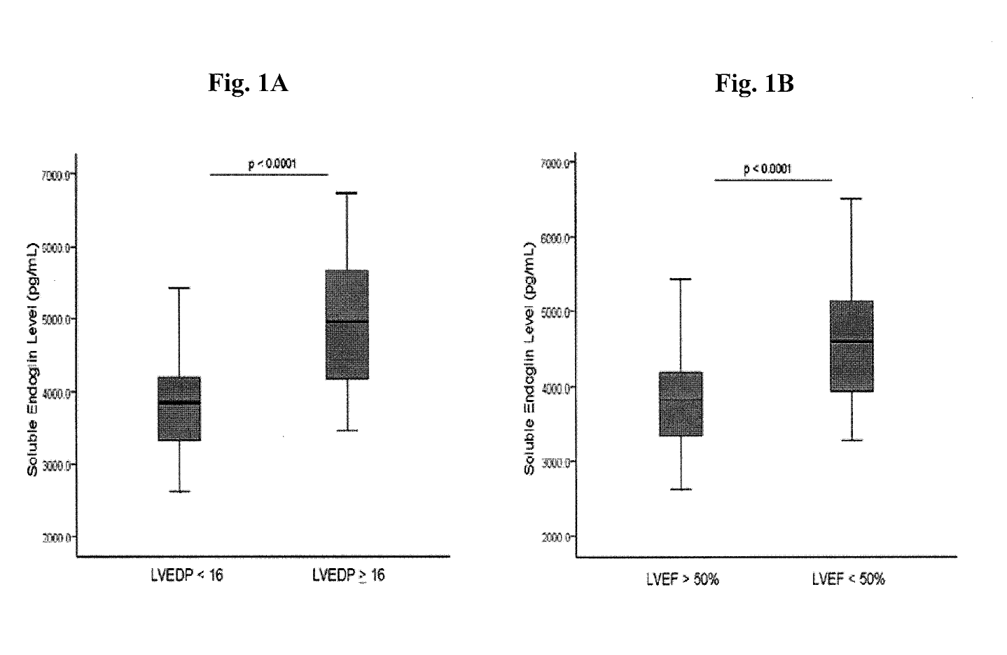

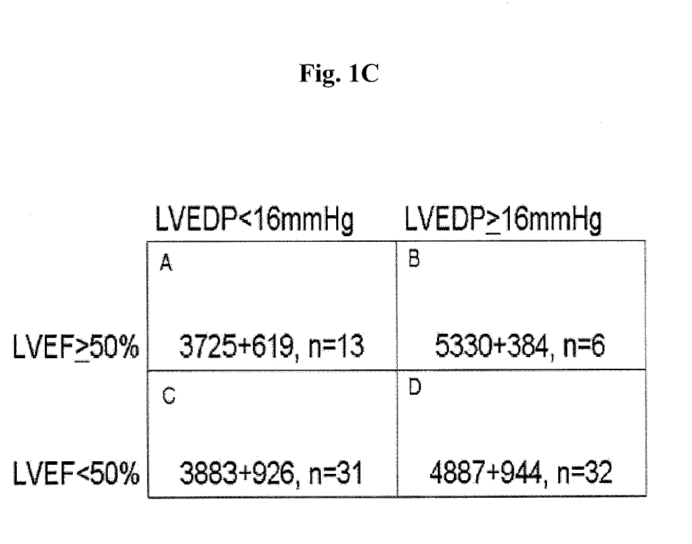

There were significant group differences in age, gender, prevalence of hypertension, smoking, diuretic use, creatinine, and blood urea nitrogen (p<0.05). Among catheterization subjects, LVEDP directly correlated with LVEF (R: −0.588; r²: 0.346; p<0.005). When categorized according to LV filling pressure, patients with a LVEDP≧16 mm Hg had significantly higher sEng levels compared to individuals with a LVEDP<16 (p<0.001; FIG. 1A). After adjusting for aforementioned group differences in age, gender, prevalence of hypertension, smoking, diuretic use, creatinine, and blood urea nitrogen with ANCOVA, sEng remained higher in patients with LVEDP≧16 compared to patients with LVEDP<16 (adjusted means: LVEDP<16=3848 pg/mL versus LVEDP≧16=4786 pg/mL; p<0.001). When grouped according to LVEF, study subjects with a low LVEF (<50%) had significantly higher sEng levels than patients with a LVEF≧50% (p<0.0001, FIG. 1B). Soluble endoglin levels correlated directly with elevated LVEDP in patients with either LVEF>50% (R=0.51, p<0.01) or LVEF<50% (R=0.50, p<0.01). When grouped by both variables, namely LVEF and LVEDP, sEng levels were significantly increased in subjects with elevated LVEDP>16, irrespective of LVEF (FIG. 1C), suggesting that levels of sEng are also elevated in patients with cardiac pressure overload and preserved LV function.

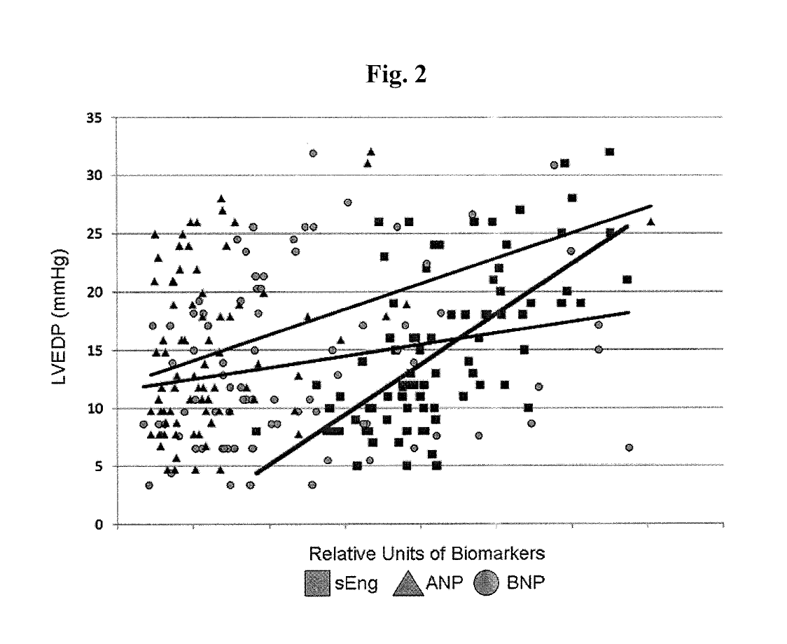

We next compared the sensitivity of sEng levels for predicting an elevated LVEDP to that of currently available biomarkers of heart failure, including ANP and BNP. Study subjects with an elevated LVEDP≧16 mm Hg exhibited increased ANP and BNP levels compared to subjects with a low LVEDP (ANP: 48±35 versus 26±14 ng/mL, p=0.01; BNP: 14±8 versus 10±7 ng/mL, p=0.03). Both ANP and BNP directly correlated with NYHA classification (ANP: R=0.298, p<0.01; BNP: R=0.309, p<0.01). Among study subjects, ANP levels correlated significantly with both LVEDP (p<0.01) and LVEF (p<0.01), while BNP levels correlated significantly with LVEDP (p=0.05), but demonstrated a weaker correlation with LVEF (p=0.09). In contrast, sEng levels exhibited a significant correlation with increased LVEDP (p<0.001) and inverse correlation with LVEF (p<0.001) (Table 2). Univariate regression plots for sEng, ANP, and BNP versus LVEDP are shown in FIG. 2. sEng levels further demonstrated a significant correlation with ANP levels (R=0.234, p=0.03); however, sEng levels did not significantly correlate with BNP levels (p>0.05) among study subjects.

TABLE 2
Univariate regression analysis of selected biomarkers as
predictors of LVEDP or LVEF

| | LVEDP | | LVEF | |
|---|---|---|---|---|
| Biomarker | R | p-value | R | p-value |
| ANP | 0.322 | 0.003 | −0.309 | 0.005 |
| BNP | 0.219 | 0.05 | −0.183 | 0.09 |
| sEng | 0.628 | <0.001 | −0.399 | <0.001 |

According to stepwise multiple regression, significant demographic predictors of LVEDP included a history of diabetes, hypertension and myocardial infarction. Overall, the model accounted for 17.7% of the variance in LVEDP. In a separate analysis, significant biomarker predictors of LVEDP included sEng (accounting for 39% of the variance) and ANP (accounting for an additional 3.2% of the variance) (Table 3). Overall, the model accounted for 43% of the variance in LVEDP. According to blockwise multiple regression, a block consisting of BNP, ANP, NYHA class, and LVEF accounted for 51% of the variance in LVEDP (p<0.001). sEng accounted for a significant incremental 10% of the variance in LVEDP above that accounted for by these traditional predictors (p<0.001).

TABLE 3
Stepwise multiple regression analysis for predictors of
LVEDP among study subjects

| | | | | 95% Confidence | |
|---|---|---|---|---|---|
| | β-coefficient | Standard | | interval (CI) | |
| | (β) | error (SE) | p-value | Lower | Upper |
| Demographic | | | | | |
| variables | | | | | |
| Diabetes | 4.060 | 1.757 | 0.024 | 0.560 | 7.560 |
| Hypertension | −4.731 | 1.632 | 0.005 | −7.984 | −1.479 |
| Myocardial | 3.892 | 1.766 | 0.031 | 0.374 | 7.410 |
| infarction | | | | | |
| Biomarker | | | | | |
| variables | | | | | |
| sEng | 4.005 | 0.601 | <0.001 | 2.810 | 5.201 |
| ANP | 0.032 | 0.015 | 0.038 | 0.002 | 0.061 |

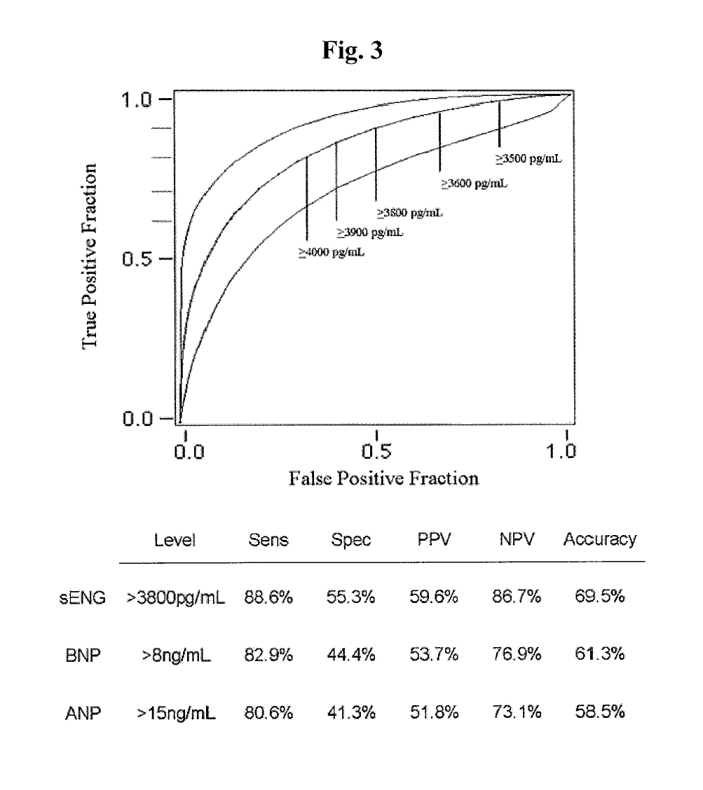

We next examined predictors of serum sEng levels among study subjects. Demographic predictors of sEng included a history of hypertension (accounting for 6.4% of the variance; β=−0.646, SE=0.231, 95% CI=−1.107-−0.185, p=0.007) and diabetes (accounting for 5.7% of the variance; β=0.556, SE=0.253, 95% CI=0.052-1.061, p=0.031). No other co-morbidity was identified as a significant predictor of sEng. With respect to medication history, diuretic use was a significant predictor of sEng (accounting for 19% of the variance; β=0.836, SE=0.262, 95% CI=0.308-1.363, p=0.003). No other medication was identified as a significant predictor of sEng. Using a receiving operative characteristic (ROC) curve, sEng levels predicted a LVEDP≧16 with an area-under-the-curve (AUC) of 0.851, exceeding the predictive value of either ANP (AUC: 0.68, p<0.01 versus sEng) or BNP (AUC: 0.65, p<0.01 versus sEng). Soluble endoglin also exhibited higher sensitivity, a greater negative predictive value, and superior accuracy compared to BNP or ANP for predicting an elevated LVEDP (FIG. 3).

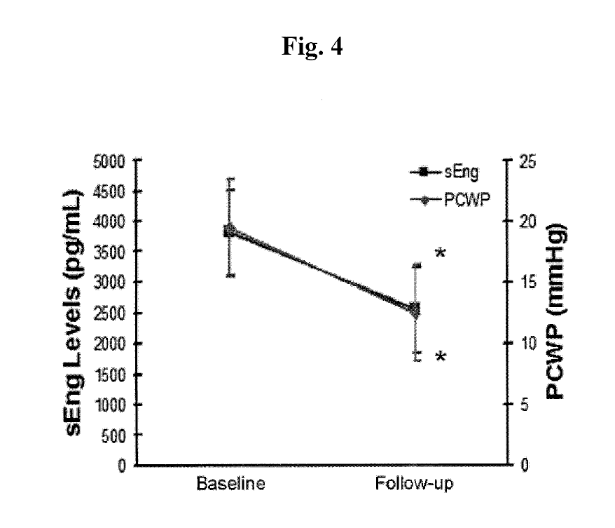

In a subgroup of 10 patients with systolic heart failure (LVEDP≧16 and LVEF<50%) receiving medical therapy for congestive heart failure in whom follow-up pulmonary artery catheter measurements were deemed clinically necessary for hemodynamic monitoring, ANOVA with repeated measures revealed a significant reduction in sEng levels after diuresis (p=0.013). Reduced sEng levels corresponded with decreased pulmonary capillary wedge pressure (PCWP) (FIG. 4), as the percent change in sEng was strongly associated with the percent change in PCWP (R=0.75, p=0.008).

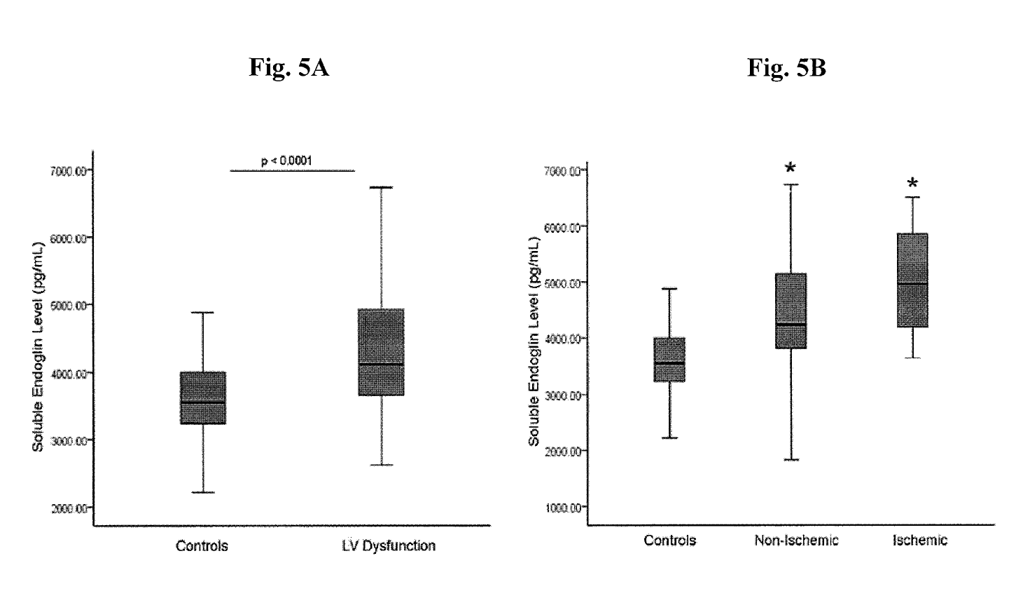

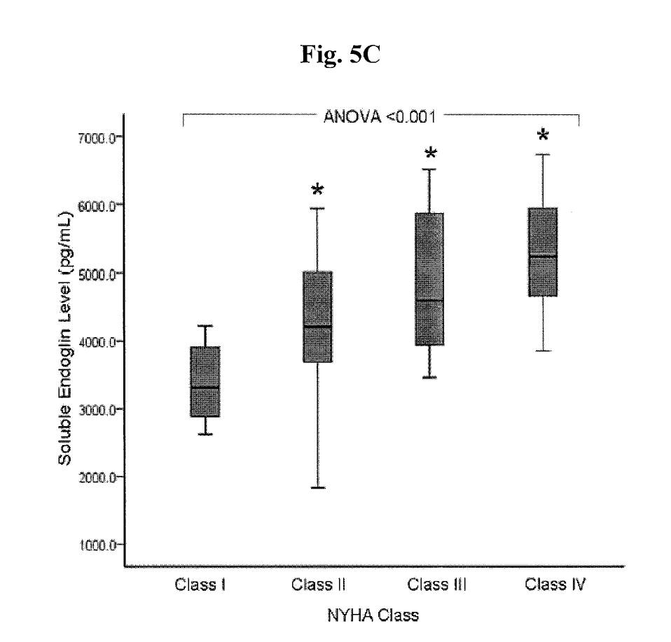

To further characterize sEng levels, twenty-five healthy volunteer subjects without any co-morbidities or currently taking any medications served as controls. Controls did not differ in age, gender, or race (p>0.05) from study subjects. Compared to healthy controls, sEng levels were significantly higher in the total group of 82 study subjects (3589±588 versus 4257±966 pg/mL, respectively, p<0.005; FIG. 5A). Soluble endoglin levels did not differ between healthy controls and patients with LVEF≧50% (3590±135 versus 3837±117 pg/mL, p=0.19) or patients with LVEDP<16 mmHg (3590±135 versus 3797±93 pg/mL, p=0.22). Among study subjects with a low LVEF<50%, sEng levels were increased regardless of whether the underlying etiology was ischemic (n=17, 4979±881 pg/mL, p<0.0001 versus controls) or non-ischemic (n=35, 4431±1000 pg/mL, p=0.004, versus controls; FIG. 5B). A trend toward higher sEng levels was observed in subjects with ischemic cardiomyopathy compared to patients with non-ischemic heart failure (p=0.06). Among study subjects, elevated sEng levels also correlated significantly with worsening NYHA classification (r=0.501, p<0.001; FIG. 5C).

### Example 2

### Mechanical Stretch Induces Endoglin Expression and sEng Release by Cardiac Fibroblasts in Congestive Heart Failure

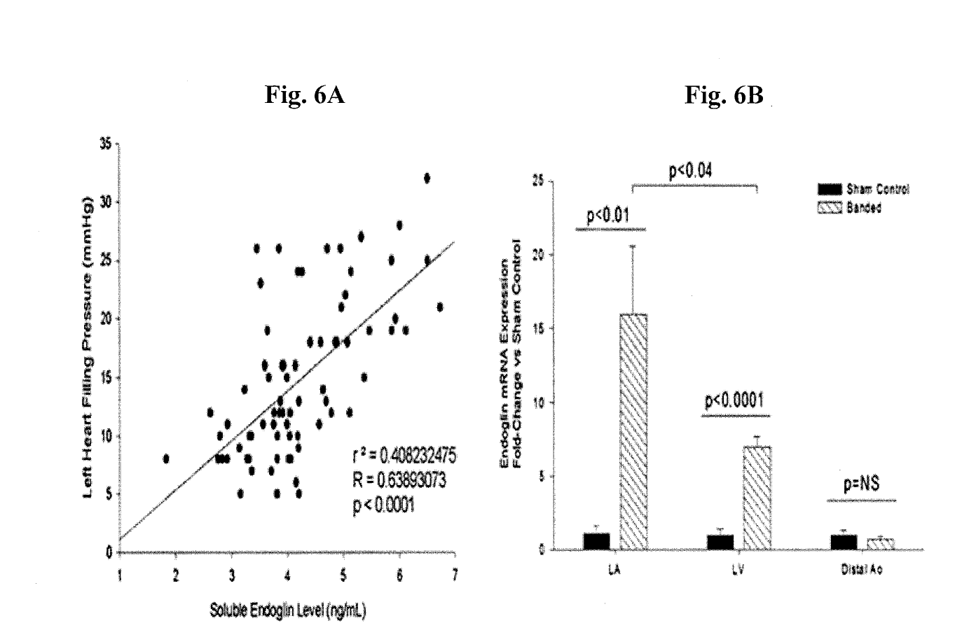

As described above, we have shown that serum levels of sEng are significantly increased in 82 human subjects with left ventricular dysfunction compared to 25 healthy, age-matched, gender-matched, and race-matched controls. Furthermore, sEng levels correlated directly with left ventricular end-diastolic filling pressure (R=0.689; p<0.0001; FIG. 6A).

Using a mouse model of cardiac pressure overload induced by thoracic aortic banding (TAC), endoglin mRNA expression was increased by 7-fold in the left ventricle (LV, p<0.001) and 14-fold in the left atrium (LA, p<0.001) compared to sham-operated controls 2 weeks after TAC (n=6/group; 10-12 week old C57/B16 male mice) (FIG. 6B). No difference in abdominal aortic endoglin expression was observed. Systemic levels of sEng were also increased in TAC mice compared to controls (1912±187 versus 1487±68 pg/mL, p=0.002) and correlated directly with LVEDP (R=0.512, p<0.001).

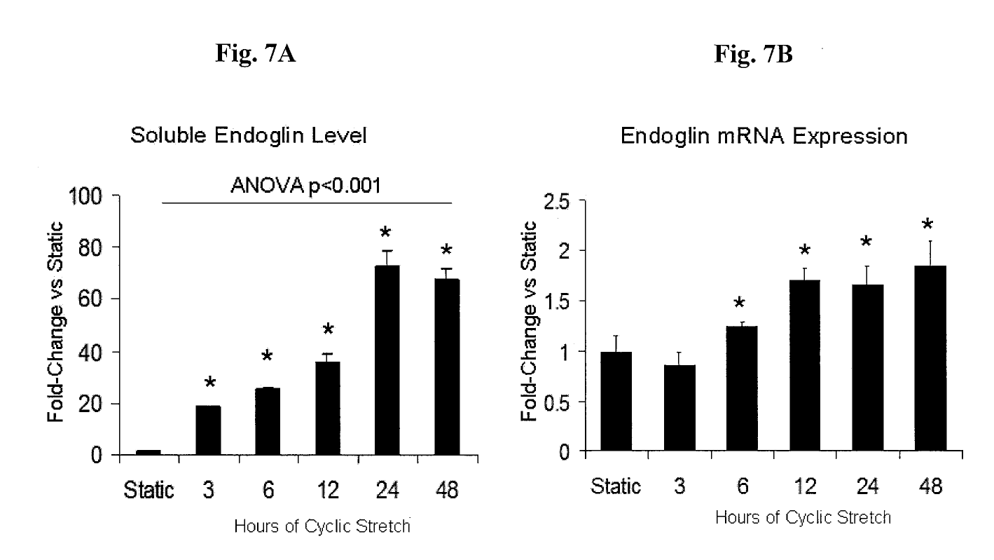

As a first step towards understanding increased endoglin expression in heart failure, we exposed cardiac fibroblasts to cyclic mechanical stretch in vitro using a FlexCell apparatus. Briefly, human cardiac fibroblasts (hCF) were cultured onto silastic membranes and exposed to 10% cyclic stretch (1 Hz) for various time points in fibroblast basal media (FBM). We identified a rapid increase in sEng release by enzyme-linked immunosorbent assay (ELISA), followed by increased endoglin mRNA expression by quantitative real-time PCR (qRT-PCR) (FIG. 7).

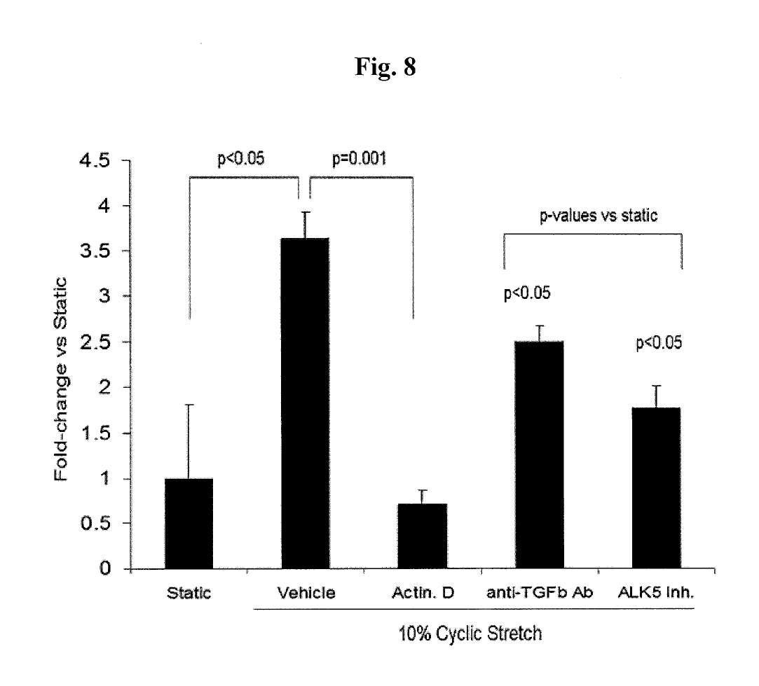

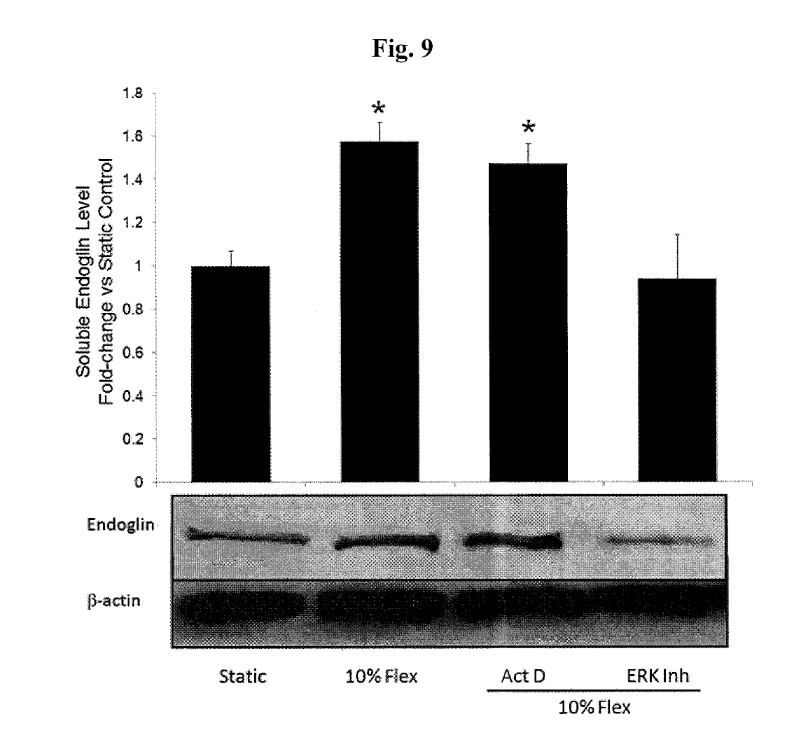

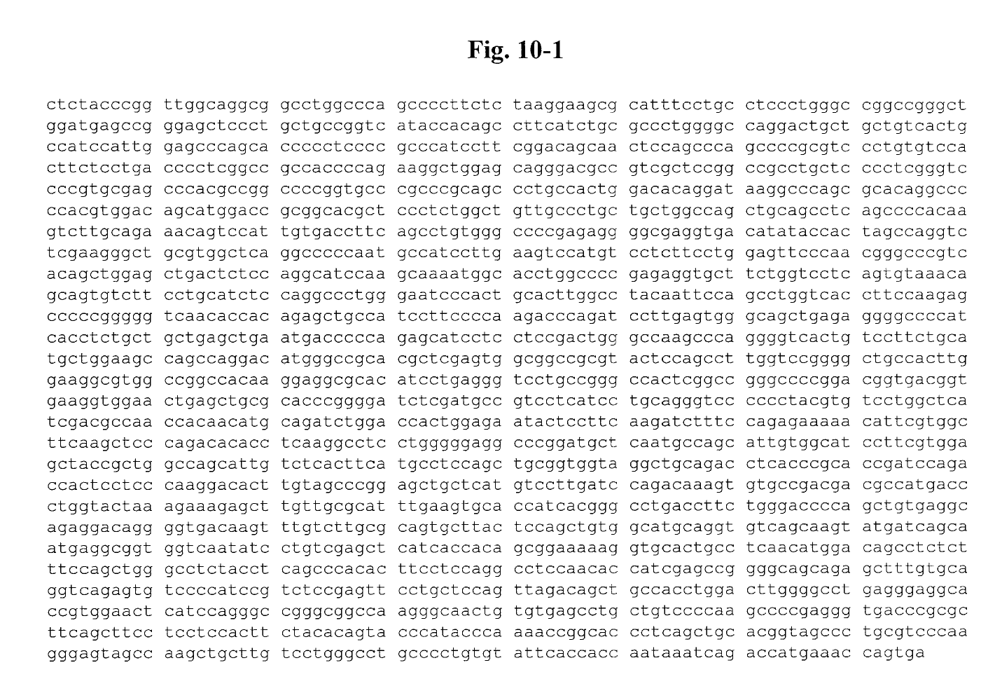

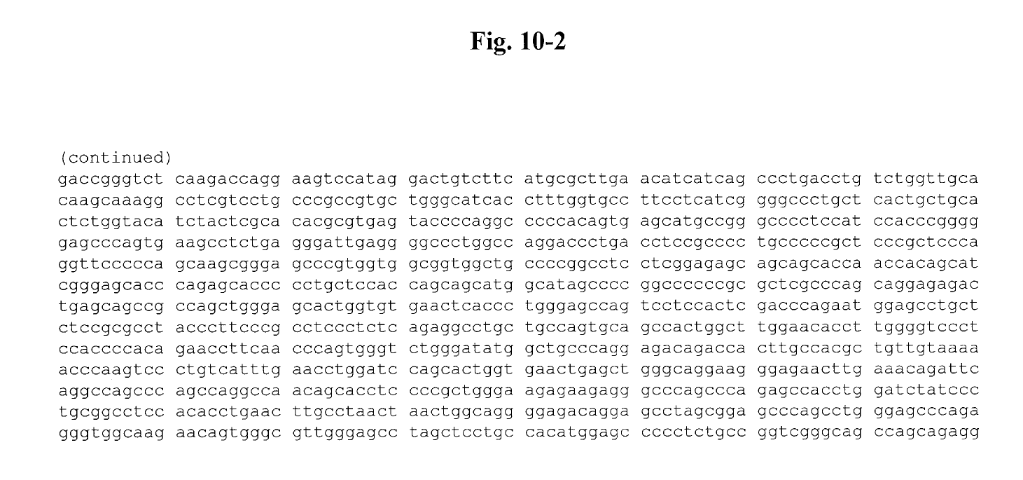

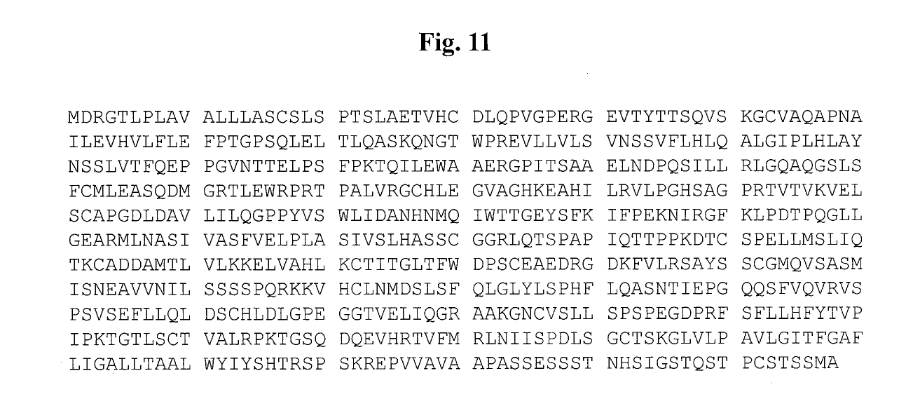

To begin exploring the mechanism underlying stretch-induced endoglin expression, we pre-treated cultured hCF in FBM with actinomycin-D (Act-D; 1 μM), SB-431542 (an ALK-5 inhibitor; 1 μM), or anti-TGFβ1 antibodies (10 ng/mL, R&D Systems) for 2 hours prior to mechanical stretch. Act-D abolished stretch-induced endoglin mRNA expression with minimal effect on sEng release or endoglin protein expression. SB-431542 or anti-TGFβ1 antibody treatment partially attenuated endoglin mRNA (mEng) expression, but failed to inhibit sEng release (FIG. 8). Furthermore, ERK-inhibition with 10 μM PD98059 attenuated stretch-induced mEng protein expression and sEng release by Western blot and ELISA (FIG. 9).

Taken together, we have shown that sEng protein release is increased in heart failure and correlates with cardiac pressure overload. Furthermore, the time course of sEng release and the use of actinoymcyin D (an inhibitor of transcription) suggest that sEng release in response to mechanical stretch does not require transcription of the endoglin gene and may instead be mediated by post-translational mechanisms such as proteolytic cleavage. Thus, sEng protein release in response to mechanical stretch occurs independently of endoglin gene transcription.

### Other Embodiments

From the foregoing description, it is apparent that variations and modifications may be made to the invention described herein to adopt it to various usages and conditions. Such embodiments are also within the scope of the following claims.

All publications, patent applications, and patents mentioned in this specification are hereby incorporated by reference to the same extent as if each independent publication or patent application was specifically and individually indicated to be incorporated by reference.

What is claimed is:

1. A method of diagnosing elevated left ventricular filling pressure (LVFP) in a subject, said method comprising measuring the level of soluble endoglin (sEng) present in a biological sample obtained from said subject, wherein an increase in the level of sEng in said subject compared to the level of sEng in a subject not suffering from elevated LVFP indicates elevated LVFP in said subject.

2. The method of claim 1, wherein said method further comprises a step of comparing the level of sEng in said sample to the a level of sEng is a sample taken from a subject not suffering from elevated LVFP or elevated RVFP.

3. The method of claim 1, wherein said level of sEng being measured is the level of sEng polypeptide or a fragment thereof.

4. The method of claim 1, wherein said elevated RVFP or LVFP occurs in a subject with a cardiovascular condition or in a subject at risk of developing a cardiovascular condition.

5. The method of claim 4, wherein said cardiovascular condition is selected from the group consisting of acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, systolic or non-systolic heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, ischemic cardiomyopathy, and peripheral vascular disease.

6. The method of claim 1, wherein said measuring comprises an immunoassay.

7. The method of claim 6, wherein said immunoassay is an enzyme-linked immunosorbent assay (ELISA), radioimmunoassay, or immunofluorescence assay.

8. The method of claim 1, further comprising measuring the level of one or more additional biomarkers in said biological sample.

9. The method of claim 8, wherein said one or more additional biomarkers are selected from the group consisting of brain natriuretic peptide (BNP), atrial natriuretic peptide (ANP), annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase.

10. The method of claim 1, wherein said biological sample is blood, serum, or plasma.

11. The method of claim 1, wherein said level of sEng in said subject not suffering from elevated left ventricular filling pressure or right ventricular filling pressure is between 1000-3500 pg/ml or wherein said level of sEng in said subject diagnosed with said elevated left ventricular filling pressure or right ventricular filling pressure is between 3500-10000 pg/ml.

12. A method of diagnosing elevated right ventricular filling pressure (RVFP) in a subject, said method comprising measuring the level of soluble endoglin (sEng) present in a biological sample obtained from said subject, wherein an increase in the level of sEng in said subject compared to the level of sEng in a subject not suffering from elevated RVFP indicates elevated RVFP in said subject.

13. The method of claim 12, wherein said method further comprises a step of comparing the level of sEng in said sample to the a level of sEng is a sample taken from a subject not suffering from elevated LVFP or elevated RVFP.

14. The method of claim 12, wherein said level of sEng being measured is the level of sEng polypeptide or a fragment thereof.

15. The method of claim 12, wherein said elevated RVFP or LVFP occurs in a subject with a cardiovascular condition or in a subject at risk of developing a cardiovascular condition.

16. The method of claim 15, wherein said cardiovascular condition is selected from the group consisting of acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, systolic or non-systolic heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, ischemic cardiomyopathy, and peripheral vascular disease.

17. The method of claim 12, wherein said measuring comprises an immunoassay.

18. The method of claim 17, wherein said immunoassay is an enzyme-linked immunosorbent assay (ELISA), radioimmunoassay, or immunofluorescence assay.

19. The method of claim 12, further comprising measuring the level of one or more additional biomarkers in said biological sample.

20. The method of claim 19, wherein said one or more additional biomarkers are selected from the group consisting of brain natriuretic peptide (BNP), atrial natriuretic peptide (ANP), annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase.

21. The method of claim 12, wherein said biological sample is blood, serum, or plasma.

22. The method of claim 12, wherein said level of sEng in said subject not suffering from elevated left ventricular filling pressure or right ventricular filling pressure is between 1000-3500 pg/ml or wherein said level of sEng in said subject diagnosed with said elevated left ventricular filling pressure or right ventricular filling pressure is between 3500-10000 pg/ml.


SEQUENCE LISTING (2 sequences)

SEQ ID NO: 1 - DNA, 3196 bp, Homo sapiens
ctctacccggttggcaggcggcctggcccagccccttctctaaggaagcgcatttcctgcctccctgggccggccgggctggatgagccgggagctccctgctgccggtcataccacagccttcatctgcgccctggggccaggactgctgctgtcactgccatccattggagcccagcaccccctccccgcccatccttcggacagcaactccagcccagccccgcgtccctgtgtccacttctcctgacccctcggccgccaccccagaaggctggagcagggacgccgtcgctccggccgcctgctcccctcgggtccccgtgcgagcccacgccggccccggtgcccgcccgcagccctgccactggacacaggataaggcccagcgcacaggcccccacgtggacagcatggaccgcggcacgctccctctggctgttgccctgctgctggccagctgcagcctcagccccacaagtcttgcagaaacagtccattgtgaccttcagcctgtgggccccgagaggggcgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagggagtagccaagctgcttgtcctgggcctgcccctgtgtattcaccaccaataaatcagaccatgaaaccagtgagaccgggtctcaagaccaggaagtccataggactgtcttcatgcgcttgaacatcatcagccctgacctgtctggttgcacaagcaaaggcctcgtcctgcccgccgtgctgggcatcacctttggtgccttcctcatcggggccctgctcactgctgcactctggtacatctactcgcacacgcgtgagtaccccaggcccccacagtgagcatgccgggcccctccatccacccgggggagcccagtgaagcctctgagggattgaggggccctggccaggaccctgacctccgcccctgcccccgctcccgctcccaggttcccccagcaagcgggagcccgtggtggcggtggctgccccggcctcctcggagagcagcagcaccaaccacagcatcgggagcacccagagcaccccctgctccaccagcagcatggcatagccccggccccccgcgctcgcccagcaggagagactgagcagccgccagctgggagcactggtgtgaactcaccctgggagccagtcctccactcgacccagaatggagcctgctctccgcgcctacccttcccgcctccctctcagaggcctgctgccagtgcagccactggcttggaacaccttggggtccctccaccccacagaaccttcaacccagtgggtctgggatatggctgcccaggagacagaccacttgccacgctgttgtaaaaacccaagtccctgtcatttgaacctggatccagcactggtgaactgagctgggcaggaagggagaacttgaaacagattcaggccagcccagccaggccaacagcacctccccgctgggaagagaagagggcccagcccagagccacctggatctatccctgcggcctccacacctgaacttgcctaactaactggcaggggagacaggagcctagcggagcccagcctgggagcccagagggtggcaagaacagtgggcgttgggagcctagctcctgccacatggagccccctctgccggtcgggcagccagcagagg

SEQ ID NO: 2 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA